(19) United States
(12) Patent Application Publication
(10) Pub. No.: US 2023/0364038 A1
(43) Pub. Date: Nov. 16, 2023

(54) ANTI-INFECTIVE DOSAGE FORMS FOR PRODUCING A NASAL RINSE

(71) Applicants: LIGHTHOUSE PHARMA GMBH; SIDROGA GESELLSCHAFT FÜR GESUNDHEITSPRODUKTE MBH

(72) Inventors: Christian FLEISCHHAMMEL, München (DE); Berthold POHL, Krailling (DE); Florian MATL, Gauting (DE); Tawfek YAZEJI, Koblenz (DE)

(21) Appl. No.: 18/029,189

(22) PCT Filed: Sep. 28, 2021
(86) PCT No.: PCT/EP2021/076671

§ 371 Date: Mar. 29, 2023

(30) Foreign Application Priority Data
Sep. 29, 2020 (DE) 20 2020 004 101.7
Oct. 5, 2020 (DE) 20 2020 004 186.6

(51) Int. Cl. A61K 31/155; A61K 9/00; A61K 31/7004; A61K 31/4425; A61K 31/444; A61K 47/36; A61K 47/32; A61K 47/38; A61K 9/08

(52) U.S. Cl. CPC A61K 31/155; A61K 9/0043; A61K 31/7004; A61K 31/4425; A61K 31/444; A61K 47/36; A61K 47/32; A61K 47/38; A61K 9/08

(57) ABSTRACT

Single-dosed, water-soluble or water-miscible drug, comprising at least one anti-infective pharmaceutical substance, for use in the local treatment of the human nasal zone.

### TECHNICAL FIELD

The present disclosure refers to a water-soluble or water-miscible drug, comprising at least one anti-infective pharmaceutical substance, for use in the local treatment of the human nasal zone and/or for producing a nasal rinse. Furthermore, the present disclosure refers to a single-dosed disposable container, comprising such a drug, the use of such a drug for producing a nasal rinse, as well as a nasal rinse, comprising at least one anti-infective pharmaceutical substance.

### BACKGROUND OF THE INVENTION

Infectious diseases are a great medical, economical, and sociopolitical challenge. The most recent example for that is the disease of the respiratory tract COVID-19 (“Corona”) caused by the SARS-CoV-2-Virus, which has claimed millions of casualties worldwide and has paralyzed entire economic areas for months. There is a high pressure worldwide search for new medicaments for treating this highly infectious disease. However, at present it cannot be foreseen when medicaments will be available which can effectively contain or fight the spreading of the SARS-CoV-2-Virus. Moreover, it is unclear who has first access to such medicaments, often elaborately developed and thus very expensive.

In view of the above mentioned, there is a need for a simple, safe, and effective solution for protection against an infection with infectious diseases (such as e.g. COVID-19), as well as for the therapeutic treatment of patients already infected.

### SUMMARY OF THE INVENTION

The problem of the present invention is to provide a drug which protects healthy persons effectively against an infection with an infectious disease, for example with the respiratory tract disease COVID-19, and which can reduce the germ load of patients already infected, particularly in the initial phase of an infection, to possibly avert a severe clinical course of disease.

According to the invention, the above-mentioned problem is solved with the features of the independent claims. Advantageous embodiments of the present invention are described in the dependent claims. Particularly, the present invention refers to a single-dosed, water-soluble or water-miscible drug, a single-dosed disposable container, the use of a single-dosed, water-soluble or water-miscible drug for producing a nasal rinse, as well as a plural-dose drug, typically in the form of a nasal spray, nasal gel, nasal drops, solution, or granulate.

The drugs according to the invention do not only have the potential to provide effective protection against a plurality of infections, in addition they can be easily produced and handled, they are cheap, and are based on the use of other drugs already used for other medical indications and classified as being compatible, for which reason sufficient safety for patients can be assumed. Furthermore, it can be assumed that the drugs according to the invention could be promptly available for broad segments of the population (also in poorer countries). The inventors act on the assumption that germs, such as e.g. SARS-CoV-2-Viruses, do not enter the lungs via the pharyngeal zone, but predominantly via the mucous membrane of the nose. The drugs according to the invention are deposited exactly at this place of entry, and try, as a type of doorman, to incapacitate germs in the human nasal zone, particularly in the nasal cavities, turbinated bones, and paranasal sinuses (to which the frontal sinuses, maxillary sinuses, and sphenoidal sinuses as well as ethmoidal cells belong). This is particularly achieved by the use according to the invention of an anti-infective nasal rinse, because same sufficiently bathes the mucous membrane of the nose exactly in these areas heavily attacked by germs. In this respect, the combination of chemical and mechanical effectiveness can—compared to conventional methods of treatment (for example, conventional nasal rinses)—lead to a more effective abatement against the germs in the nasal zone. The single-dosed drug according to the invention can be converted in a simple and user-friendly manner into the corresponding dosage form (nasal rinse), which results in a good compliance. In doing so, the single-dosed drug according to the invention has such a concentration of a pharmaceutical substance which allows preparing the anti-infective-acting nasal rinse according to the invention which is physiologically compatible and unproblematic for the user simply by adding tap water, but at the same time effectively fights against the germs load in the nasal zone. A concerted balance between—on the one hand—the physiological compatibility and harmlessness for the user, and—on the other hand—a sufficient effectiveness against the relevant germs of the nasal zone are in the specific focus of the inventors. These and other advantages will be revealed to the skilled person in the course of the detailed description of the present invention.

Thus, according to an aspect, a water-soluble or water-miscible drug is suggested which comprises at least one anti-infective pharmaceutical substance, and which can be used in the local treatment of the human nasal zone. Preferably, the drug is single-dosed.

According to a further aspect, a water-soluble or water-miscible drug is proposed, which comprises at least one anti-infective pharmaceutical substance, and which is suitable for the preparation of a nasal rinse. Preferably, the drug is single-dosed.

According to a further aspect, the use of a water-soluble or water-miscible form of a drug for producing a nasal rinse is suggested, wherein the nasal rinse preferably exists in the form of an aqueous or aqueous-organic, isotone (isotonic), slightly hypertone or hypertone (hypertonic) solution. Preferably, the drug is single-dosed.

According to a further aspect, the use of a water-soluble or water-miscible drug in a device for introducing a liquid into the human nasal zone is suggested, preferably in a nasal douche. Preferably, the drug is single-dosed.

According to a further aspect, a single-dosed disposable container is suggested, which comprises a single-dosed, water-soluble or water-miscible drug, and which is preferably selected from the group consisting of sachet, ampulla, bag and syringe.

According to a further aspect, a nasal rinse is suggested, which comprises at least one anti-infective pharmaceutical substance.

According to a further aspect, a plural-dose drug is suggested which comprises at least one anti-infective pharmaceutical substance, wherein the anti-infective pharmaceutical substance has a molar mass of 75 g/mol to 750 g/mol.

According to a further aspect, a kit is suggested, which comprises at least two of the following components:
a water-soluble or water-miscible drug according to the invention for preparing a nasal rinse;
a plural-dose drug according to the invention for preparing a nasal rinse;
a single-dosed, water-soluble or water-miscible drug according to the invention for preparing a mouth rinse;
a (conventional) mouth rinse; and/or a device for introducing a liquid dosage form into the nasal zone, preferably a nasal douche.

Further features and advantages of the present invention will be presented in the following extensive description of the preferred embodiments of the forms of drugs.

### DETAILED DESCRIPTION

Even if in the following preferred embodiments are described, the scope of protection of the invention is not limited to depicted embodiments, but also comprises similar embodiments.

In the following, reference is made to different embodiments of the invention in a more detailed way.

According to an aspect of the present invention, a water-soluble or water-miscible drug is provided. The drug can be single-dosed. The drug can comprise at least one anti-infective pharmaceutical substance, and can be preferably provided for use in the local treatment of the human nasal zone.

In the context of the present invention, drug means a preparation consisting of a drug and optionally added auxiliary agents. Sometimes it is necessary to convert a drug before use in a suitable dosage form (for example a dry juice, which due to stability problems is converted to a suspension only before use). Because, however, this is not mandatory, in the context of the present invention the term “dosage form” can be used synonymously to the term “drug”.

Single-dosed drug means in the context of the present invention a form of a drug which comprises a form of a pharmaceutical substance in an amount of a pharmaceutical substance (dose), which is administered once to a patient at a defined point of time to achieve the desired therapeutic effect. A water-soluble drug in the context of the present invention means a drug which dissolves in water (i.e. which is “soluble” in water), preferably “easily” or “very easily” dissolves in water, according to the definition of the European Pharmacopeia at 15° C. to 25° C. Insofar, a substance is presumed to be “soluble” in water if at 15° C. to 25° C. 1 g of this substance is dissolved in 10 to 30 ml of water. Accordingly, a substance is presumed to be “easily soluble” in water if at 15° C. to 25° C. 1 g of this substance dissolves in 1 to 10 ml of water, and as “very easily soluble” if at 15° C. to 25° C. 1 g thereof dissolves in less than 1 ml of water. A water-soluble drug in the context of the present invention means a drug which mixes with water completely by forming a single homogenous phase.

The single-dosed drug according to the invention has such characteristics that it is suitable for the preparation of a nasal rinse. Thus, the drug according to the invention for example dissolves easily and well in water, comprises the anti-infective drug (and optionally auxiliary agents) preferably in such a concentration that the nasal rinse produced therefrom is both physiologically acceptable (e.g. isotone or slightly hypertone, respectively, and does not irritate the mucous membrane) and is also sufficiently effective for its use in the local treatment of the human nasal zone. In the following, the requirements of the drug according to the invention are discussed in more detail.

The single-dosed drug according to the invention can be a solid, semi-solid or liquid drug. A solid drug can be selected from the group consisting of powder, granulate, and tablet. As a semi-solid drug it can for example comprise a gel. As a liquid drug, it is preferably available in form of an aqueous, aqueous-organic, or organic solution, which particularly preferably is hypertone. If present, the organic component of such a solution can be selected from the group of alcohols, and particularly can be ethanol, propanol and/or glycerin, particularly preferred glycerin. Preferably, the aqueous, aqueous-organic, or organic solution is hypertone, i.e. it has a higher osmotic pressure than the blood plasma.

Particularly preferably, the drug according to the invention is a solid form of a drug, particularly a granulate or a powder. Specifically preferred, the drug according to the invention is a granulate. Using granulates as a drug according to the present invention has different advantages compared to powders or solutions. The granulates according to the invention have better flow properties as powder, for which reason they can be better filled or metered. In addition, the granulates according to the invention can be applied easier compared to powder, inter alia because they produce less dust and they dissolve better in an aqueous medium, which can result in a better compliance. Compared to solutions, the granulates according to the invention have the advantage that even more unstable pharmaceutical substances can be packed in drugs, without having to use additional auxiliary agents (for example preserving agents or light-stability agents) to prevent the already dissolved pharmaceutical substance from decomposing.

The single-dosed, water-soluble or water-miscible drug of the present invention comprises at least one anti-infective pharmaceutical substance. Particularly, the drug according to the present invention can comprise one, two, three, four, five, six or seven, preferably one or two, particularly preferred two anti-infective pharmaceutical substances. The use of a combination of two or more pharmaceutical substances in the present case has the advantage that each of the pharmaceutical substances used can be used in a smaller concentration, because the total concentration of the pharmaceutical substances used is relevant for the efficiency of a drug. Depending on the pharmaceutical substance, a smaller concentration can contribute substantially to a better compatibility. This is particularly of importance for the very sensitive mucous membrane of the nose. However, with a combination of pharmaceutical substances it must be guaranteed that they are sufficiently compatible with each other. The inventors of the present application, based on extensive experiments, have succeeded in identifying this fine line between compatibility and efficiency of anti-infective pharmaceutical substances (and preferably of combinations of pharmaceutical substances) in the nasal zone, and to provide a drug having the potential to prevent diseases in the nasal zone and/or to fight them effectively.

A pharmaceutical substance within the meaning of this application is a substance which is used in the preparation of a drug (medicament) as a component which is active as a pharmaceutical ingredient (i.e. pharmacologically active). Thus, pharmaceutical substances are those components of a drug which are the cause for its (for example pharmacologic) effectiveness. Mostly, the pharmaceutical substance is processed to the drug in combination with one or more pharmaceutical auxiliary agents, but occasionally also without auxiliary agents. The pharmacologically inactive ingredients of a drug are called auxiliary agents. Examples of auxiliary agents are preservatives, fillers, dyes etc. It is to be considered that in different drugs the same substance can be seen as a pharmaceutical substance (i.e. as an active ingredient) or as an auxiliary agent (i.e. as a non-active ingredient). For example, chlorohexidine is used in eye and nose medicaments (for example nasal sprays) as preservative, and thus as an auxiliary agent. On the contrary, in mouth rinses chlorohexidine is used as the pharmaceutical substance (i.e. as active ingredient), primarily to kill germs. If the present invention refers to an anti-infective pharmaceutical substance, then the (pharmacologically) active component of the claimed drug is meant, i.e. that component, which is the cause for its (pharmacological) effectiveness (anti-infective effect). Accordingly, the anti-infective pharmaceutical substance of the present invention is an active component of the drug according to the invention.

The concentration of the at least one anti-infective pharmaceutical substance in the water-soluble or water-miscible (and typically single-dosed) drug, and particularly in the granulate (i.e. the entire concentration of all pharmaceutical substances used in the drug according to the invention) is adjusted such that the nasal rinse to be produced therefrom is isotonic, slightly hypertone or hypertone. Particularly, the concentration of the at least one anti-infective pharmaceutical substance in the single-dosed, water-soluble or water-miscible drug according to the invention can be 0.5 weight-% to 40 weight-%, preferably 0.8 weight-% to 20 weight-%, more preferred 1 weight-% to 15 weight-%, and particularly preferred 3 weight-% to 12 weight-%, alternatively especially preferred 0.5 weigh-% to 12 weight-%, relative to the total weight of the single-dosed, water-soluble or water-miscible drug.

The water-soluble or water-miscible (and typically single-dosed) drug can comprise at least one auxiliary agent. If present, the concentration of the at least one auxiliary agent (i.e. the total concentration of all auxiliary agents used in the form of a drug according to the invention) in the single-dosed, water-soluble or water-miscible form of a drug according to the invention, particularly in the granulate, can be 60 weight-% to 99.5 weight-%, preferably 80 weight-% to 99.2 weight-%, particularly preferred 85 weight-% to 99 weight-%, and especially preferred 88 weight-% to 97 weight-%, alternatively especially preferred 88 weight-% to 99.5 weight-%, relative to the total weight of the single-dosed, water-soluble or water-miscible form of a drug.

Suitable auxiliary agents can be selected from the group consisting of water, seawater, NaCl, mineral salt mixture, dexpanthenol, sesame oil, hyaluronic acid, carrier substances, such as e.g. cellulose derivatives (particularly hydroxypropylmethyl cellulose (HPMC), hydroxypropyl cellulose (HPC), hydroxyethyl cellulose (HEC), Na-carboxymethyl cellulose (Na-CMC), sugar alcohols (for example mannitol), gum arabic, polyvinylpyrrolidone (PVP), saccharose, glucose, agents for masking flavor, and combinations thereof.

Particularly preferred auxiliary agents in the drug according to the invention can be selected from the group consisting of one or more cellulose derivatives (particularly hydroxypropylmethyl cellulose (HPMC), hydroxypropyl cellulose (HPC), hydroxyethyl cellulose (HEC), Na-carboxymethyl cellulose (Na-CMC)), sugar alcohols (particularly mannitol), gum arabic, polyvinylpyrrolidone (PVP), and combinations thereof, and comprise particularly a cellulose derivative (particularly methyl cellulose) and/or mannitol. Using a cellulose derivate (particularly methyl cellulose) in combination with a sugar alcohol (particularly mannitol) has turned out to be particularly advantageous, particularly with respect to their compatibility with the used at least one anti-infective pharmaceutical substance, as well as with respect to the behavior of dissolving the drug in water.

With anti-infective pharmaceutical substances in the context of the present invention, compounds are meant which are used and effective as so-called anti-infective agents against microorganisms, particularly bacteria, viruses, fungi, and/or protozoans.

The anti-infective pharmaceutical substance of the drug according to the invention can have a molar mass of at most 1000 g/mol, preferred 75 g/mol to 750 g/mol, particularly preferred 100 g/mol to 600 g/mol, particularly 300 g/mol to 600 g/mol. According to preferred embodiments, the anti-infective pharmaceutical substance of the drug according to the invention has no peptide bond, and thus is no amino acid, polypeptide or protein. According to further preferred embodiments, the anti-infective pharmaceutical substance of the drug according to the invention is not based on the anti-infective effect of a metal (such as e.g. silver ions etc.), and typically has no herbal origin. Concerning this matter, it is to be noted that the inventors have concentrated on pharmaceutical substances having a greater potential of effectiveness as prophylaxis against and/or for abatement of viral and/or bacterial diseases of the human nasal zone as is basically described for herbal anti-infective agents.

The anti-infective pharmaceutical substance of the drug according to the invention can be selected from the group consisting of antibiotics, antituberculosis agents, antifungal agents, antiviral pharmaceutical substances, antiseptics, and combinations thereof.

Preferred antibiotics for use in a drug according to the invention are selected from the group consisting of aminoglycosides, carbapenemes, cephalosporins, fluorochinolone, glycopeptides, and lipoglycopeptides (for example Vanomycin), ketolides (for example Telithromycin), macrolide (for example Erythromycin), monobactames (Aztreonam), and combinations thereof. Particularly preferred antibiotics comprise locally effective antibiotics, such as e.g. Tyrothricin, preferably in combination with Lactoferrin and Lysozyme. Antituberculosis agents for use in the drug according to the invention can be selected from the group consisting of Isoniazid, Rifampicin, Rifabutin, Ethambutol, and Pyrazinamide, antifungal agents, and combinations thereof. Antifungal agents for use in the drug according to the invention can be selected from the group consisting of 5-fluorocytosin, Amphotericin B, Naftifin, Sertaconazol, Ciclopirox, Nystatin, and combinations thereof.

According to a particularly preferred embodiment, the anti-infective pharmaceutical substance of the present invention is an antiviral pharmaceutical substance. With an antiviral pharmaceutical substance in the context of the present invention, a pharmaceutical substance is meant which is virucidal and/or virustatic.

Substances are called viruzids or virizde (“killing virus”) which reduce or completely prohibit the infectivity of viruses by destruction of the viral nucleic acid, more often by denaturation or removal of the viral envelope. The capability for this inactivation of the virus is called virucidic behavior. Viruzids which can inactivate only viruses with an envelope are described as being restricted viruzidic. Substances which inhibit the propagation of viruses are called virostatic agents.

According to a particularly preferred embodiment, the anti-infective pharmaceutical substance of the present invention is effective against viruses with an envelope, particularly against the SARS-CoV-2-Virus.

According to a particularly preferred embodiment, the anti-infective pharmaceutical substance is an antiseptic pharmaceutical substance (antiseptic).

Preferred antiviral pharmaceutical substances and/or preferred antiseptics of the drug according to the present invention can be selected from the group consisting of acyclic alcohols, quaternary ammonium compounds, compounds containing iodine, halogenated compounds, quinoline derivatives, benzoquinone derivatives, phenol derivatives, and combinations thereof.

Preferred acyclic alcohols for use in the drug according to the invention can be selected from the group consisting of ethanol, hexanol, n-propanol, and isopropanol, and combinations thereof.

Preferred quaternary ammonium compounds for use in the drug according to the invention can be selected from the group consisting of benzalkonium, benzethonium chloride, Brilliant Green, Cetrimid, cetylpyridinium chloride, octenidin(-dihydrochloride), polyhexanid, and combinations thereof.

Preferred compounds containing iodine for use in the drug according to the invention can be selected from the group consisting of povidone-iodine, iodine tincture, and combinations thereof.

Preferred halogenated compounds for use in the drug according to the invention can be selected from the group consisting of Triclosan, chlorohexidine, 2,4-dichlorobenzyl alcohol, and combinations thereof.

A preferred quinoline derivative for use in the drug according to the invention is oxiquinoline.

A preferred benzoquinone derivative for use in the drug according to the invention is ambazon.

Preferred phenol derivatives for use in the drug according to the invention can be selected from the group consisting of Triclosan, hexachlorophene, and combinations thereof.

According to a particularly preferred embodiment, the antiviral and/or antiseptic pharmaceutical substance can be selected from the group consisting of octenidin(-dihydrochloride), chlorohexidine, Triclosan, dequalinium chloride, cetylpyridinium chloride, hexamine-dii sethionate, amylmetakresol, 2,4-dichlorobenzyl alcohol, benzalkonium chloride, and combinations thereof. Particularly preferred antiviral and/or antiseptic pharmaceutical substances can be selected from the group consisting of octenidin(-dihydrochloride), chlorohexidine, Triclosan, cetylpyridinium chloride, and combinations therof.

Particularly preferred combinations of pharmaceutical substances of two pharmaceutical substances comprise the combination of chlorohexidine and cetylpyridinium chloride, the combination of octenidin(-dihydrochloride) and chlorohexidine, as well as the combination of cetylpyridinium chloride and octenidin(-dihydrochloride), and particularly the combination of chlorohexidine and cetylpyridinium chloride. The inventors of the present invention have particularly found out that exactly the use of one or more antiseptic(s), and particularly the combination of a halogenated compound (particularly chlorohexidine) and a quaternary ammonium compound (particularly cetylpyridinium chloride) has both the potential to fight effectively against the germ spectrum in the nasal zone, as well as has proven for the user to be extremely user friendly and compatible.

The water-soluble or water-miscible (and typically single-dosed) drug according to the invention typically is provided for use in the local treatment of the human nasal zone. With the human nasal zone in the context of the present invention typically at least one area is meant selected from the group of nasal cavities, turbinated bones, and paranasal sinuses (inclusive of frontal sinuses, maxillary sinuses, and sphenoidal sinuses, as well as ethmoidal cells). With a local treatment of the human nasal zone, it is meant in the context of the present invention typically the topical application of the drug according to the invention (which preferably is converted into the dosage form according to the invention before use) on the mucous membrane of the nose of at least one of the above-mentioned areas of the nasal zone. Using the drug according to the invention particularly comprises use for prophylaxis (prevention) against an infection with a pathogenic organism, preferably with bacteria and/or viruses and/or fungi, particularly preferred as prophylaxis (prevention) against an infection with viruses, particularly with the SARS-CoV—Virus (particularly especially SARS-CoV-2-Virus).

Furthermore, using the drug according to the invention can comprise the use in the treatment of a disease in the human nasal zone, preferably induced by bacteria and/or viruses and/or fungi, particularly preferably in the treatment of a disease induced by viruses, particularly the SARS-CoV-Virus, particularly especially by the SARS-CoV-2-Virus, thus particularly in the treatment of COVID-19.

Further exemplary diseases of the human nasal zone against which the drug according to the invention can be used are typically irritations of the mucous membrane in the nose, also in the context of the metabolic disease mucoviscidosis, and/or inflammatory diseases of the paranasal sinuses, also in the context of the metabolic disease mucoviscidosis. These diseases often occur as a concomitant phenomenon of common colds, influenzal infects, a COVID-19 disease, but also as independent diseases.

A further aspect of the present invention refers to the use of the single-dosed, water-soluble or water-miscible drug according to the invention for the preparation of a nasal rinse, wherein the nasal rinse preferably is available in the form of an aqueous or aqueous-organic, isotone, slightly hypertone or hypertone solution. With slightly hypertone in the context of the present invention, a solution is meant having an osmolarity comparable to a >0.9 weight-% to 3 weight-% NaCl solution. Thus, the single-dosed, water-soluble or water-miscible drug according to the invention can be converted before use into a liquid dosage form. Accordingly, the present invention also refers to using the single-dosed, water-soluble or water-miscible drug in a device for bringing a liquid in the human nasal zone, preferably in a nasal douche.

According to a further aspect of the present invention, a single-dosed disposable container is provided containing the single-dosed, water-soluble or water-miscible drug according to the invention. The disposable container preferably can be selected from the group consisting of sachet (side sealed bag), ampulla, bag and syringe. Preferably, the disposable container can be an ampulla or a sachet.

An ampulla according to the invention can comprise an ampulla wall surrounding a cavity, and at least one opening in the ampulla wall which is closed liquid tight by a removable lock. In the cavity, preferably the single-dosed, water-miscible drug in form of an aqueous, aqueous-organic or organic solution is located, which preferably is hypertone. If maximally filled with the solution, the ampulla typically has a weight of 1 g to 15 g, preferably 1.5 g to 12 g, particularly preferred 2.0 to 7 g. The ampulla according to the invention has a maximal volume expansion which is characterized in that the ampulla can be completely surrounded by an imaginary hollow sphere having a radius of at most 10 cm, preferably at most 7 cm. Typically, there is no point of contact and/or point of intersection between the surface of the sphere and the ampulla. The cavity of the ampulla typically has a volume of 0.5 ml to 15 ml, preferably 1.5 ml to 12 ml, particularly preferred 2.0 g to 7 g. The wall of the ampulla can comprise plastic or aluminum. Typically, the plastic is selected from the group consisting of PE, PET, PVC, PS, PP, PA and/or PC, preferably PE and/or PET. In doing so, the wall of the ampulla can be constructed from plastic, preferably PE and/or PET, or from aluminum, or it can consist of the aforementioned materials.

A sachet according to the invention can comprise a sachet wall surrounding a cavity which typically is sealed air-tight. In the cavity, preferably the single-dosed, water-soluble or water-miscible drug according to the invention is located in the form of a powder, granulate, solution (particularly an aqueous, aqueous-organic, or organic solution), or a gel, preferably of a granulate. If maximally filled with the single-dosed, water-soluble or water-miscible drug according to the invention inclusive optional additives (such as e.g. NaCl), the sachet typically has a weight of 1 g to 15 g, preferably 1.5 g to 12 g, particularly preferred 2.0 g to 7 g, alternatively particularly preferred 0.3 g to 7 g. The sachet according to the invention has a maximal volume expansion which is characterized in that the sachet can be completely surrounded by an imaginary hollow sphere having a radius of at most 15 cm, preferably at most 10 cm. Typically, there is no point of contact and/or point of intersection between the surface of the sphere and the sachet. The cavity of the sachet typically has a volume of 0.5 ml to 15 ml, preferably 1.5 ml to 12 ml, particularly preferred 2.0 g to 7 g. The wall of the sachet can comprise a composite film made of plastic or aluminum. Typically, the wall of the sachet comprises a polyester-aluminum-polyethylene-laminate or a paper-aluminum-polyethylene-laminate, or can consist of one of those laminates.

The single-dosed, water-soluble or water-miscible drug according to the invention, and particularly the single-dosed granulate according to the invention, typically has a weight of 100 mg to 5000 mg, preferably 200 mg to 3000 mg, particularly preferred 300 mg to 2000 mg. Hence, the single-dosed, water-soluble or water-miscible drug according to the invention, and particularly the single-dosed granulate according to the invention has a weight of the pharmaceutical substance (total weight of all pharmaceutical substances) of 0.5 mg to 2000 mg, preferably 1.6 mg to 600 mg, particularly preferred 9 mg to 240 mg, and particularly 12 mg to 100 mg.

According to a further aspect of the present invention, a nasal rinse is described comprising at least one of the anti-infective pharmaceutical substances described above, and which is provided for use in the local treatment of the human nasal zone described above. Typically, the nasal rinse according to the invention is isotone, slightly hypertone, or hypertone. This can be achieved in that already the amount of the single-dosed, water-soluble or water-miscible drug (particularly granulate) used for the preparation of the nasal rinse according to the invention is sufficient to make the nasal rinse solution isotone, slightly hypertone, or hypertone. Alternatively, by admixing additives (for example NaCl), the solution can be made isotone, slightly hypertone, or hypertone. Accordingly, the nasal rinse according to the invention can comprise further additives, particularly NaCl, in addition to the above-mentioned pharmaceutical substances and/or auxiliary agents. Typically, the nasal rinse according to the invention can comprise an additive (particularly NaCl) in a concentration of 0.1 weight-% to 10 weight-%, preferably 0.5 weight-% to 8 weight-%, particularly preferred 0.8 weight-% to 3 weight-%, in relation to the total weight of the nasal rinse. Typically, the nasal rinse according to the invention is an aqueous or organic-aqueous solution. The nasal rinse according to the invention can be used in the form of a spray. Accordingly, the aqueous or organic-aqueous solution can be nebulized by means of a spray can.

According to an aspect of the present invention, the concentration of the at least one anti-infective pharmaceutical substance in the nasal rinse according to the invention (i.e. the total concentration of all pharmaceutical substances used in the nasal rinse according to the invention) can be 0.01 weight-% to 3 weight-%, preferably 0.05 weight-% to 1 weight-%, particularly preferred 0.08 weight-% to 0.25 weight-%, in relation to the total weight of the nasal rinse. Particularly preferred, the concentration of the at least one anti-infective pharmaceutical substance in the nasal rinse according to the invention (i.e. the total concentration of all pharmaceutical substances used in the nasal rinse according to invention) can be 0.003 weight-% to 0.09 weight-%, preferably 0.005 weight-% to 0.08 weight-%, particularly 0.008 weight-% to 0.05 weight-%, in relation to the total weight of the nasal rinse. The inventors surprisingly could determine that this concentration of the pharmaceutical substance is substantially below the dosage of the mouth rinses available on the market, which is about 0.1 or 0.2 weight-%. This could be attributed to the increased sensitivity of the mucous membrane of the nose compared to the mouth mucosa. At the same time, however, one can act on the assumption of a sufficient effectiveness, because the at least one anti-infective pharmaceutical substance of the present invention even in very low concentrations still has a sufficient effectiveness.

The nasal rinse according to the invention can comprise at least one of the above described auxiliary agents. If present, the concentration of the at least one auxiliary agent (i.e. the total concentration of all auxiliary agents used in the nasal rinse) can be 0.05 weight-% to 20 weight-%, preferably 0.08 weight-% to 15 weight-%, particularly preferred 0.1 weight-% to 2 weight-%, relative to the total weight of the nasal rinse.

The nasal rinse according to the invention can be used in a device for placing a liquid in the human nasal zone, typically in a nasal douche. A nasal rinse shall replace the mechanical cleaning function of the low flow of liquid on the mucous membrane of the nose present under physiological conditions (ciliated epithelium transports liquid and particles). If this cleaning function is impaired (e.g. by a disease of the nasal zone) pathogens (for example viruses) can easier penetrate into the cells of the mucous membrane of the nose, and infect same. For adults, the cleaning flow rate when using a Tillable nasal douche typically is between 200 and 300 ml or 200 and 500 ml, while for children until twelve years between 100 ml and 150 ml are sufficient. The nasal rinse according to the invention can be used one to three times a day, particularly two times a day. The nasal rinse according to the invention can have a volume of 50 ml to 350 ml, preferably 80 ml to 320 ml, particularly preferred 100 to 300 ml. According to an embodiment, the nasal rinse according to the invention can be single-dosed. Thus, for example the nasal rinse according to the invention can be comprised in a single-dosed disposable container.

According to a further aspect, the present invention relates to a plural-dose drug which at least comprises one of the above-described anti-infective pharmaceutical substances, typically with a molar mass of 75 g/mol to 750 g/mol, particularly 300 g/mol to 600 g/mol, and which is preferably used in the above-described local treatment of the human nasal zone. The plural-dose drug according to the invention can be typically available in the form of a nasal spray, nasal gel or nasal drops. Typically, the plural-dose drug according to the invention in the form of a nasal spray, nasal gel or nasal drops is isotone or hypertone. The concentration of the at least one anti-infective pharmaceutical substance in the plural-dose drug according to the invention in the form of a nasal spray, nasal gel or nasal drops (i.e. the total concentration of all pharmaceutical substances used in the plural-dose form of a drug according to the invention) can be 0.001 weight-% to 3 weight-%, preferably 0.005 weight-% to 1 weight-%, particularly preferred 0.008 weight-% to 0.25 weight-%, and especially preferred 0.01 to 3 weight-%, particularly 0.01 weight-% to 0.1 weight-%, relative to the total weight of the plural-dose of the drug. Particularly preferred, the concentration of the at least one anti-infective pharmaceutical substance in the plural-dose drug according to the invention (i.e. the total concentration of all pharmaceutical substances used in the plural-dose drug according to the invention) can be 0.003 weight-% to 0.09 weight-%, preferably 0.005 weight-% to 0.08 weight-%, particularly 0.008 weight-% to 0.05 weight-%, relative to the total weight of the plural-dosed drug.

Alternatively, the plural-dose drug according to the invention can be a granulate or a water-miscible, aqueous, organic-aqueous or organic solution which comprises at least one of the above-described anti-infective pharmaceutical substances, and which can be used for the preparation of the nasal rinse according to the invention. The concentration of the at least anti-infective pharmaceutical substance in the plural-dose drug (i.e. the total concentration of all pharmaceutical substances used in the plural-dose drug according to the invention) is adjusted such that the nasal rinse to be manufactured from a single-dose of the plural-dose drug is isotone, slightly hypertone, or hypertone (≥290±10 mOsmol/kg). Particularly, the concentration of the at least one anti-infective pharmaceutical substance in the plural-dose drug according to the invention can be 0.5 weight-% to 40 weight-%, preferably 0.8 weight-% to 20 weight-%, particularly preferred 1 weight-% to 15 weight-%, and especially preferred 3 weight-% to 12 weight-%, alternatively especially preferred 0.5 weight-% to 12 weight-%, relative to the total weight of the plural-dose drug. If present, the concentration of the at least one auxiliary agent (i.e. the total concentration of all auxiliary agents used in the drug according to the invention) in the plural-dose drug according to the invention, particularly in the granulate, can be 60 weight-% to 99.5 weight-%, preferably 80 weight-% to 99.2 weight-%, particularly preferred 85 weight-% to 99 weight-%, and especially preferred 88 weight-% to 97 weight-%, alternatively especially preferred 88 weight-% to 99.5 weight-%, relative to the total weight of the plural-dose drug.

The plural-dose drug according to the invention can have, in the form of an aqueous, organic-aqueous, or organic solution, a volume of 100 ml to 1500 ml, preferably 200 ml to 1000 ml, particularly preferred 300 to 750 ml. The plural-dose drug according to the invention can comprise, in the form of a granulate, a mass of 10 g to 1000 g, preferably 20 g to 800 g, particularly preferred 50 g to 500 g. The plural-dose drug according to the invention can comprise, both in the form of a granulate or a water-miscible, aqueous, organic-aqueous, or organic solution, a number of 20 to 200 singles doses, preferably 30 to 100, particularly preferred 40 to 80.

According to a further aspect of the present invention, a single-dosed, water-soluble or water-miscible drug is suggested which comprises at least one of the above-described anti-infective pharmaceutical substances and the above-described optional auxiliary agents, and particularly is used in the local treatment of the human mouth and nasal zones and/or is suitable for the preparation of a mouth rinse. The concentration of the at least one infective pharmaceutical substance in the single-dosed, water-soluble or water-miscible drug (i.e. the total concentration of all pharmaceutical substances used in this drug according to the invention) is adjusted such that the mouth rinse resulting therefrom can have a concentration of the pharmaceutical substance (i.e. the total concentration of all pharmaceutical substances used in the mouth rinse according to the invention) of 0.025 weight-% to 0.5 weight-%, preferably 0.05 weight-% to 0.3 weight-%, particularly preferred 0.08 weight-% to 0.25 weight-%, relative to the total weight of the mouth rinse. Therefore, the concentration of the at least one anti-infective pharmaceutical substance in the single-dosed, water-soluble or water-miscible form of a drug for the production of a mouth rinse can be 0.025 weight-% to 0.5 weight-%, preferably 0.05 weight-% to 0.3 weight-%, particularly preferred 0.08 weight-% to 0.25 weight-%, relative to the total weight of the single-dosed form of the drug. The drug according to the invention can be a solid, semi-solid or liquid drug. As a solid form of a drug, it can be selected from the group consisting of powder, granulate and tablet. As a semi-solid drug it can, for example, comprise a gel. As a liquid drug, it is preferably available in the form of an aqueous or aqueous-organic solution. Preferably, the aqueous or aqueous-organic solution is isotone, slightly hypertone, or hypertone. Particularly preferred, the drug according to the invention is available as granulate, powder, or in the form of an aqueous or aqueous-organic, hypertone solution, particularly as a granulate.

According to a further aspect of the present invention, a kit is provided which comprises at least two of the following components:
a water-soluble or water-miscible drug according to the invention for preparing a nasal rinse;
a plural-dosed drug according to the invention for preparing a nasal rinse;
a single-dosed, water-soluble or water-miscible drug according to the invention for preparing a mouth rinse;
a (conventional) mouth rinse; and/or
a device for introducing a liquid dosage form into the nasal zone, preferably a nasal douche.

The use of a nasal rinse according to the invention in combination with a mouth rinse according to the invention renders possible a protection against infections for almost the entire upper respiratory tract (mouth, nose, and pharynx) in a simple, flexible, and cost-efficient manner.

The production of the drug according to the invention can be carried out by methods known to the man skilled in the art. Thus, principally the production of solid drugs (such as for example powder, granulate or tablet), of semi-solid drugs (such as for example gel), as well as liquid drugs (such as for example aqueous, aqueous-organic or organic solutions) is known to the skilled man, and sufficiently described in the specialist literature, for example in the textbook “Bauer/Frömming/Führer—Pharmazeutische Technologie, 10^{th}, revised edition 2017, Wissenschaftliche Verlagsgesellschaft Stuttgart”.

For example, the drug according to the invention in the form of a granulate can be prepared by means of dry granulation or wet granulation (e.g. fluidized bed granulation). Dry granulation and wet granulation, respectively, are well-established methods for preparing granulates.

Particularly preferred embodiments refer to the following aspects which can be combined with the above-mentioned embodiments.
Aspect 1. Granulate, particularly single-dosed granulate, for the production of a nasal rinse, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use as prophylaxis against a disease in the nasal zone, particularly against an infection with a virus and/or a bacterium, particularly the SARS-CoV-Virus.
Aspect 2. Granulate, particularly single-dosed granulate, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use in the treatment of a disease in the nasal zone, particularly in the treatment of a bacterial and/or viral disease of the nasal zone, particularly SARS-CoV.
Aspect 3. Granulate, particularly single-dosed granulate, comprising, relative to the total weight of the granulate, 3 weight-% to 12 weight-% of at least one anti-infective pharmaceutical substance, selected from the group consisting of octenidin(-dihydrochloride), chlorohexidine, cetylpyridinium chloride, Triclosan, and combinations thereof, particularly 3 weight-% to 12 weight-% of a combination of chlorohexidine and cetylpyridinium chloride, for use in the treatment of a disease of the nasal zone, particularly in the treatment of a bacterial and/or viral disease of the nasal zone.
Aspect 4. Granulate, particularly single-dosed granulate, comprising, relative to the total weight of the granulate, 3 weight-% to 12 weight-% of a combination of chlorohexidine and cetylpyridinium chloride, and 88 weight-% to 97 weight-% of a combination of one or more sugar alcohol(s) and one or more cellulose derivative(s), for use in the treatment of a disease of the nasal zone, particularly in the treatment of a bacterial and/or viral disease of the nasal zone.
Aspect 5. Single-dosed, organic-aqueous or organic solution for the production of a nasal rinse, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use as prophylaxis against a disease in the nasal zone, particularly against an infection with a virus, particularly the SARS-CoV-Virus.
Aspect 6. Organic-aqueous or organic plural-dose solution for the preparation of a nasal rinse, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use as prophylaxis against a disease in the nasal zone, particularly against an infection with a virus and/or a bacterium, particularly the SARS-CoV-Virus.
Aspect 7. Use of a granulate, particularly of a single-dosed granulate, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for the production of a nasal rinse.
Aspect 8. Nasal rinse, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use as prophylaxis against a disease of the nasal zone, particularly against an infection with a virus, particularly the SARS-CoV-Virus.
Aspect 9. Nasal rinse, comprising at least one anti-infective pharmaceutical substance, selected from the group consisting of octenidin(-dihydrochloride), chlorohexidine, cetylpyridinium chloride, Triclosan, and combinations thereof, particularly a combination of chlorohexidine and cetylpyridinium chloride, for use in the treatment of a disease of the nasal zone, particularly for use in the treatment of a bacterial and/or viral disease of the nasal zone, particularly SARS-CoV.

Although the present invention has been described above by means of typical examples, it is not limited thereon, but modifiable in a variegated manner. Furthermore, the invention is not limited to the application possibilities referred to.

### EXAMPLES

Several exemplary anti-infective granulates (see Table 1) have been prepared by means of wet granulation.

### Formulation for the Preparation of Anti-Infective Granulates G1 to G10

TABLE 1

| Ingredients | G1 | G2 | G3 | G4 | G5 |
|---|---|---|---|---|---|
| Mannitol | 20.10 g | 22.40 g | 20.90 g | 20.40 g | 20.05 g |
| Chlorohexidine | 2.50 g | — | — | — | — |
| diacetate (CHX) | | | | | |
| Cetylpyridinium | — | 2.05 g | — | 5.20 g | |
| chloride (CPC) | | | | | |
| Octenidin(-dihydrochloride) | — | — | 2.50 g | — | 5.10 g |
| Gum Arabic GA) | 0.58 g | 0.60 g | 0.60 g | — | — |
| Kollidon | — | — | — | 0.60 g | — |
| (Polyvinylpyrrolidone) | | | | | |
| Methyl cellulose (MC) | — | — | — | — | 0.03 g |
| Purified H₂O | 2.93 g | 2.99 g | 3.01 g | 3.02 g | 2.97 g |
| Ingredients | G6 | G7 | G8 | G9 | G10 |
| Mannitol | 20.05 g | 22.44 g | 20.85 g | 22.30 g | 22.36 g |
| Chlorohexidine | 2.51 g | 1.05 g | 2.55 g | 1.00 g | 1.05 g |
| diacetate (CHX) | | | | | |
| Cetylpyridinium | 1.25 g | 1.00 g | 1.00 g | 1.00 g | 1.00 g |
| chloride (CPC) | | | | | |
| Gum Arabic GA) | 0.58 g | 0.60 g | 0.60 g | — | — |
| Kollidon | — | — | — | 0.60 g | — |
| (Polyvinylpyrrolidone) | | | | | |
| Methyl cellulose (MC) | — | — | — | — | 0.03 g |
| Purified H₂O | 2.94 g | 2.97 g | 3.03 g | 3.00 g | 2.99 g |

### Protocol of Production (in Laboratory Scale)

Providing a mortar with pestle,
Closing pores of the mortar with an indifferent auxiliary agent,
Providing an amount of a pharmaceutical substance to be dispersed in the mortar (if applicable, comminute with flow adjustment medium),
Comminute with about the same amount of the auxiliary agent,
Stepwise adding further amounts of auxiliary agents and comminute,
Weighing purified water and providing in a beaker,
Weighing Gum Arabic, Kollidon (PVP) or methyl cellulose and carefully spreading on the provided water and allowing it to soak for at least 30 min, if applicable stirring and slightly heating until a clear solution is generated,
Adding now dropwise the granulating liquid to the powder mixture and blending until a snowball-like mass develops,
Sieving the mass through a sieve, for example pore size 2000, and drying in the drying cabinet, e.g. at 40° C., until mass constant is reached)*¹,
Sieving again the granulate (pore size 1400), and subsequently filling in single-dosed bags. *1 The remnant water moisture of the granulates was not determined. However, it is to be assumed that by means of the drying process the water portion in the finished granulate can be neglected when calculating the weight proportion of the pharmaceutical substance(s) relative to the total weight of the produced granulate.

### In Vitro and In Vivo Studies (Results See Table 2)

It was tried to dissolve 0.342 g of each granulate G1 to G10 in 250 ml water each at room temperature by shaking. In doing so, the time for dissolving was determined, and the solutions L1 to L10 produced were subsequently examined with respect to their foaming behavior and a possible precipitate formed (visual determination). All in all, for each granulate G1 to G10, ten solutions were prepared (i.e. 10 solutions for G1, 10 solutions for G2, etc.). The dissolving behavior of the granulates G1 to G10 was evaluated according to the following scheme:

### Dissolving Behavior

3 ≙poor (dissolving time: >1 min, heavy foaming, precipitate formed)
2 ≙medium (dissolving time: 10 seconds to 1 min, slightly foaming, no precipitate)
1 ≙good (dissolving time: <10 seconds, no foaming, no precipitate)

In order to get an isotone or slight hypertone, respectively, ready-to-use nasal rinse solution N to each of solutions L1 to L10 about 3 g Eimer® Nasenspülsalz (produced 2021) was added. Subsequently, each of the ready-to-use nasal rinse solutions N1 to N10 thus produced by means of a nasal douche was tested at a proband (compatibility test). All in all, each ready-to-use nasal rinse solution N1 to N10 was tested with 10 probands each (per proband, 1 test per day). The probands could evaluate the ready-to-use solution according to the following scheme (compatibility assessment):

### Compatibility Assessment

3 ≙poor (irritation of the mucous membrane of the nose, slight redness of the pharyngeal posterior wall, dryness sensation in the pharynx up to 2 h after application)
2 ≙medium (no irritations of the mucous membrane of the nose, no redness of the pharynx, slight dryness sensation in the pharynx up to 30 min after application)
1 ≙good (no irritations of the mucous membrane of the nose, no redness in the pharynx, no dryness sensation in the pharynx, neutral feeling after application)

The results with respect to dissolving behavior and compatibility are summarized in Table 2 below. For the average evaluation of each granulate and each nasal rinse solution, respectively, for each granulate (i.e. G1, G2, G3, . . . ) and each nasal rinse solution (i.e. N1, N2, N3, . . . ), respectively, the following rating was introduced:
Σ(10 samples)≤14 ≙good ≙“O”
Σ(10 samples)>14≤23 ≙medium ≙“Δ”
Σ(10 samples)>23≤30=≙poor ≙“X”

Thus, for the evaluation of the dissolving behavior of the granulates, a granulate (for example G1) can achieve a minimum of 10 points (best evaluation) and a maximum of 30 points (poorest evaluation). The same applies for the evaluation of the compatibility of the nasal rinse solutions N1 to N10.

TABLE 2

| | | Compatibility of |
|---|---|---|
| | Solution | the nasal rinse |
| Samples | behavior | solutions produced |
| Gx/Lx/Nx | (G1 to G10) | (N1 to N10) |
| 1 | ◯ | Δ |
| 2 | ◯ | Δ |
| 3 | ◯ | Δ |
| 4 | Δ | X |
| 5 | Δ | X |
| 6 | ◯ | Δ |
| 7 | ◯ | ◯ |
| 8 | Δ | Δ |
| 9 | ◯ | ◯ |
| 10 | ◯ | ◯ |

1. A single-dosed, water-soluble or water-miscible drug for the production of a nasal rinse, comprising at least one anti-infective pharmaceutical substance for use in the local treatment of the human nasal zone.

2. The single-dosed, water-soluble or water-miscible drug according to claim 1, wherein the drug is used as prophylaxis against a bacterial and/or viral disease of the nasal zone and/or in the treatment of a bacterial and/or viral disease of the nasal zone.

3. The single-dosed, water-soluble or water-miscible drug according to claim 1, wherein the drug is a solid drug, selected from the group consisting of powder, granulate, and tablet.

4. The single-dosed, water-soluble or water-miscible drug according to claim 1 wherein the at least one anti-infective pharmaceutical substance in the drug is contained in a concentration of 0.8 weight-% to 20 weight-%, relative to the total weight of the drug.

5. The single-dosed, water-soluble or water-miscible drug according to claim 1, wherein the anti-infective pharmaceutical substance is selected from the group consisting of antibiotics, antituberculosis agents, antifungal agents, antiviral pharmaceutical substances, antiseptics, and combinations thereof.

6. The single-dosed, water-soluble or water-miscible drug according to claim 1, wherein the anti-infective pharmaceutical substance is selected from the group consisting of acyclic alcohols, quaternary ammonium compounds, compounds containing iodine, halogenated compounds, quinoline derivatives, benzoquinone derivatives, phenol derivatives, and combinations thereof.

7. The single-dosed, water-soluble or water-miscible drug according to claim 1, wherein the anti-infective pharmaceutical substance is an antiviral pharmaceutical substance which is effective against viruses with envelope.

8. The single-dosed, water-soluble or water-miscible drug according to claim 1, wherein the anti-infective pharmaceutical substance has a molar mass of 300 g/mol to 600 g/mol.

9. The single-dosed, water-soluble or water-miscible drug according to claim 1, wherein the drug is a granulate, and wherein the drug comprises one or two anti-infective pharmaceutical substances.

10. The single-dosed, water-soluble or water-miscible drug according to claim 1, wherein the drug comprises one or more auxiliary agents, selected from the group consisting of cellulose derivative, sugar alcohol, gum arabica, polyvinylpyrrolidone (PVP), and combinations thereof.

11. A single-dosed disposable container, comprising a single-dosed, water-soluble or water-miscible drug according to claim 1.

12. The single-dosed, water-soluble or water-miscible drug according to claim 1, wherein the drug is used in preparing a nasal rinse.

13. The single-dosed, water-soluble or water-miscible drug according to claim 1, wherein the drug is used in a device for introducing a liquid into the human nasal zone.

14. A nasal rinse, comprising at least one anti-infective pharmaceutical substance, wherein the anti-infective pharmaceutical substance is selected from the group consisting of acyclic alcohols, quaternary ammonium compounds, compounds containing iodine, halogenated compounds, quinoline derivatives, benzoquinone derivatives, phenol derivatives, and combinations thereof.

15. The nasal rinse according to claim 14, wherein the at least one anti-infective pharmaceutical substance is contained in the nasal rinse in a concentration of 0.003 weight-% to 0.09 weight-%, relative to the total weight of the nasal rinse.

16. The single-dosed, water-soluble or water-miscible drug according to claim 7, wherein the anti-infective pharmaceutical substance is an antiviral pharmaceutical substance which is effective against the SARS-CoV-2-Virus.

17. The single-dosed disposable container of claim 11, wherein the disposable container is selected from the group consisting of a sachet, ampulla, bag, and syringe.

18. The single-dosed, water-soluble or water-miscible drug according to claim 12, wherein the nasal rinse is available in the form of an aqueous or aqueous-organic, isotone or hypertone solution.

19. The single-dosed, water-soluble or water-miscible drug according to claim 13, wherein the device comprises a nasal douche.
